(19) United States
(12) Patent Application Publication
(10) Pub. No.: US 2024/0426774 A1
(43) Pub. Date: Dec. 26, 2024

(54) OXYGEN SENSOR, WATER QUALITY MEASURING DEVICE AND OXYGEN MEASURING METHOD

(71) Applicant: HORIBA Advanced Techno, Co., Ltd., Kyoto-shi, Kyoto (JP)

(72) Inventors: Kensuke TODA, Kyoto-shi (JP); Yuichiro KOMATSU, Kyoto-shi (JP)

(21) Appl. No.: 18/684,372

(22) PCT Filed: Aug. 17, 2022
(86) PCT No.: PCT/JP2022/031052

§ 371 Date: Feb. 16, 2024

(30) Foreign Application Priority Data
Aug. 27, 2021 (JP) 2021-138710

(51) Int. Cl. G01N 27/404; G01N 27/416; G01N 33/18

(52) U.S. Cl. CPC G01N 27/404; G01N 27/416; G01N 33/18

(57) ABSTRACT

An oxygen sensor comprises electrolyte, a liquid-containing portion that has an opening and that contains the electrolyte therewithin, a permeable membrane that has oxygen permeability and that covers the opening, and positive electrode and negative electrode that are arranged so as to come in contact with the electrolyte, the negative electrode contains tin, and the electrolyte contains polyol.

### TECHNICAL FIELD

This application relates to an oxygen sensor, a water quality measuring device and an oxygen measuring method.

### BACKGROUND ART

Conventionally, an oxygen sensor might, for example, comprise electrolyte, a liquid-containing portion within which the electrolyte is contained, a permeable membrane which has oxygen permeability, and positive and negative electrodes which are arranged so as to come in contact with the electrolyte (e.g., Patent Reference No. 1). In addition, at an oxygen sensor associated with Patent Reference No. 1, the negative electrode thereof contains tin.

Because the negative electrode does not contain cadmium, mercury, lead, or the like, this makes it possible to reduce the environmental impact thereof. It so happens that there are cases in which oxygen sensors are, for example, used to measure the dissolved oxygen of a target liquid which is at low temperature. And particularly when measuring dissolved oxygen at target liquids at temperatures of 0° C. or lower, there is a possibility that freezing of the electrolyte will make it impossible to measure the dissolved oxygen therewithin.

### CITATION LIST

### Patent Literature

Patent Reference No. 1: JP A 2006-194708

### SUMMARY OF THE INVENTION

### Technical Problem

The problem is therefore to provide an oxygen sensor, water quality measuring device, and oxygen measuring method that will make it possible to properly measure the dissolved oxygen in a target liquid which is at a low temperature.

### Solution to Problem

There is provided an oxygen sensor comprising:
electrolyte;
a liquid-containing portion that has an opening and that contains the electrolyte therewithin;
a permeable membrane that has oxygen permeability and that covers the opening; and
positive electrode and negative electrode that are arranged so as to come in contact with the electrolyte;
wherein the negative electrode contains tin; and
wherein the electrolyte contains polyol.

There is provided a water quality measuring device comprising the oxygen sensor.

There is provided an oxygen measuring method wherein the oxygen sensor is used to measure dissolved oxygen at a target liquid.

### BRIEF DESCRIPTION OF THE DRAWINGS

FIG. 1 is a full view of a water quality measuring device associated with an embodiment.

FIG. 2 is a sectional end view of section II-II in FIG. 1.

FIG. 3 is an enlarged view of region III in FIG. 2.

FIG. 4 is a potential-pH diagram for an Sn—H₂O system (temperature=25° C.).

### DESCRIPTION OF EMBODIMENTS

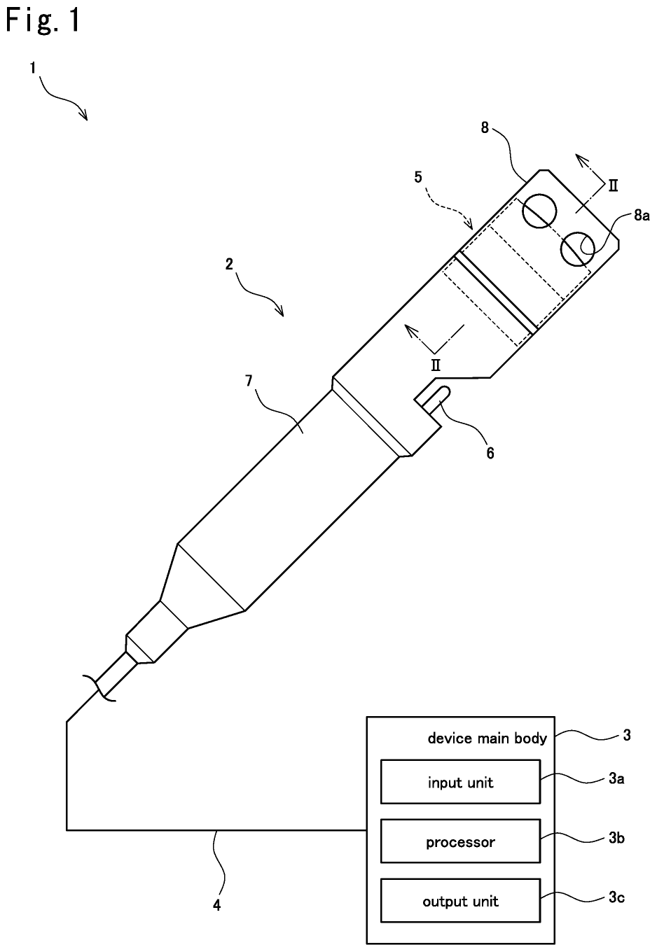

Below, embodiments of an oxygen sensor and a water quality measuring device are described with reference to FIG. 1 through FIG. 4. At the respective drawings, note that dimensional ratios in the drawings and actual dimensional ratios are not necessarily consistent, and note further that dimensional ratios are not necessarily consistent from drawing to drawing.

As shown in FIG. 1, water quality measuring device 1 might, for example, comprise detector 2 which detects water quality of a target liquid that is a target for measurement; device main body 3 which is capable of communicating with detector 2; and communication means 4 which is capable of causing communication between detector 2 and device main body 3. Device main body 3 may, e.g., as is the case in the present embodiment, comprise input unit 3a at which information is input; processor 3b which processes information; and output unit 3c at which information is output.

Communication means 4 might, for example, be wired communication means (e.g., a cable) as is the case in the present embodiment; or it might, for example, be wireless communication means. Note that detector 2 and device main body 3 may be constituted in integral fashion.

While there is no particular limitation with respect thereto, input unit 3a might, for example, be button(s), touch panel(s), and/or the like. In addition, information in the form of an instruction to begin measurement or the like might, for example, be input at input unit 3a.

Processor 3b might, for example, comprise a CPU, MPU, and/or other such processor(s); ROM, RAM, and/or other such memory or memories; various interfaces, and so forth. More specifically, processor 3b might, for example, comprise an acquisition unit which acquires information from detector 2, input unit 3a, and/or the like; a storage unit which stores information; an arithmetic unit which performs arithmetic operations with respect to information (e.g., water quality values); and a control unit which controls various unit(s) (e.g., output unit 3c ) of water quality measuring device 1.

While there is no particular limitation with respect thereto, output unit 3c might, for example, be display device(s) and/or the like. Note that output unit 3c might, for example, be transmission means that outputs (transmits) signal(s) to the exterior of water quality measuring device 1. In addition, output unit 3c might output results of measurement (e.g., water quality values) and/or the like.

Detector 2 comprises sensor(s) 5, 6 that detect the water quality of a target liquid. More specifically, detector 2 comprises at least oxygen sensor 5 which detects dissolved oxygen at a target liquid. In addition, detector 2 might, for example, comprise at least one other sensor 6 that detects water quality item(s) (e.g., pH, electrical conductivity, turbidity, temperature, and/or the like) other than dissolved oxygen; or it might, for example, comprise only oxygen sensor(s) 5. While there is no particular limitation with respect thereto, in accordance with the present embodiment, detector 2 comprises oxygen sensor 5, and temperature sensor 6 which detects the temperature of the target liquid.

Detector 2 might, for example, comprise detector main body 7 to which respective sensors 5, 6 are attached; and protective unit 8 which protects oxygen sensor 5. A constitution may be adopted in which, e.g., as is the case in the present embodiment, respective sensors 5, 6 are arranged at the tip of detector 2. This will make it possible for a person who wishes to carry out measurement to grasp detector main body 7 and cause the tip of detector 2, i.e., respective sensors 5, 6, to be immersed within the target liquid, as a result of which detection of the water quality of the target liquid is made to occur.

A constitution might, for example, be adopted in which, to protect oxygen sensor 5, protective unit 8 is attached to detector main body 7 in such fashion as to cover oxygen sensor 5. In addition, a constitution may be adopted in which, e.g., as is the case in the present embodiment, protective unit 8 is formed (e.g., from metal) so as to have rigidity in order to prevent deformation thereof, and such that it comprises an opening 8a for entry thereinto by the target liquid. Note that it is also possible to adopt a constitution in which protective unit 8 is, for example, formed so as to be film-like and so as to have elasticity.

Furthermore, while there is no particular limitation with respect thereto, temperature sensor 6 may, e.g., as is the case in the present embodiment, be arranged at a location which is recessed with respect to detector main body 7. This will make it possible, e.g., where detector 2 is used in such fashion that it is thrown into a river or the like, to suppress occurrence of a situation in which temperature sensor 6 might otherwise hit the bottom or bank of the river.

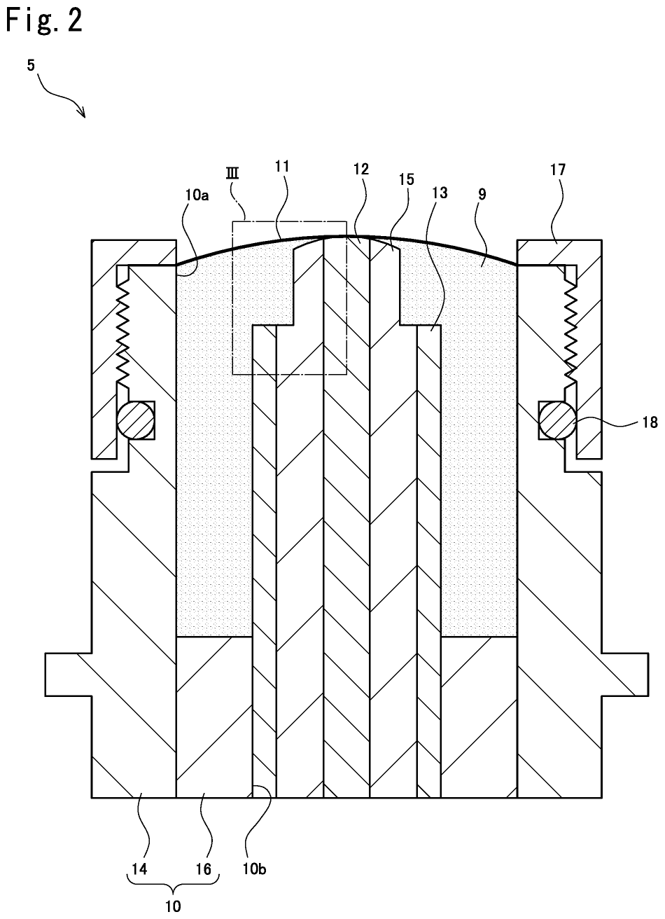

As shown in FIG. 2, oxygen sensor 5 comprises electrolyte 9; liquid-containing portion 10 within which electrolyte 9 is contained and which has opening 10a at the tip thereof; permeable membrane 11 which has oxygen permeability and which covers opening 10a of liquid-containing portion 10; and positive electrode 12 and negative electrode 13 which are arranged so as to come in contact with electrolyte 9. In addition, negative electrode 13 contains tin.

While there is no particular limitation with respect thereto, negative electrode 13 might, for example, be formed by machining it from tin, extrusion molding of tin, plating or vapor deposition of tin onto the surface of an electrically conductive member, or the like. From the standpoint of the fact that the greater the tin content the longer will be its service life, machining from tin or extrusion molding of tin is more preferred than plating or vapor deposition of tin onto the surface of an electrically conductive member.

Furthermore, while there is no particular limitation with respect thereto, in accordance with the present embodiment, oxygen sensor 5 is such that application of a voltage by means of an external power supply or the like between positive electrode 12 and negative electrode 13 is unnecessary, as it is a galvanic cell-type sensor for which an electric potential is spontaneously produced between positive electrode 12 and negative electrode 13. And by measuring the electric potential which is produced between positive electrode 12 and negative electrode 13, it will be possible to measure dissolved oxygen.

Permeable membrane 11 is a membrane that permits passage therethrough of oxygen but does not permit passage therethrough of liquids. While there is no particular limitation with respect thereto, permeable membrane 11 might, for example, be a polyethylene membrane or a tetrafluoroethylene-hexafluoropropylene copolymer (FEP) or other such fluororesin membrane or the like. Furthermore, while there is no particular limitation with respect thereto, thickness of permeable membrane 11 might, for example, be 12.5 μm to 50 μm.

Oxygen sensor 5 may, e.g., as is the case in the present embodiment, comprise housing 14 which is formed so as to be cylindrical; retainer 15 which is arranged at the interior of housing 14 and which retains positive electrode 12 and negative electrode 13; electrode securing portion 16 which causes positive electrode 12 and negative electrode 13 to be secured to housing 14; and membrane securing portion 17 which secures permeable membrane 11 by causing an outside circumferential portion of permeable membrane 11 to be sandwiched between it and housing 14.

While there is no particular limitation with respect thereto, in accordance with the present embodiment, liquid-containing portion 10 is made up of housing 14 and electrode securing portion 16. In addition, electrolyte 9 is contained within the interior of liquid-containing portion 10 by permeable membrane 11. While there is no particular limitation with respect thereto, note in the present embodiment that liquid-containing portion 10 comprises opening 10b at the base portion thereof, and that opening 10b is closed due to presence of positive electrode 12, negative electrode 13, and retainer 15.

In addition, at least a portion of each of positive electrode 12 and negative electrode 13 is arranged within the interior of liquid-containing portion 10. Note that oxygen sensor 5 might, for example, comprise a seal (e.g., O-ring) 18 to prevent entry of the target liquid into the interior of liquid-containing portion 10 from the space between membrane securing portion 17 and housing 14.

At oxygen sensor 5, oxygen within the target liquid passes through permeable membrane 11, and after having passed through permeable membrane 11 the oxygen is reduced at positive electrode 12, which causes occurrence of an electrochemical reaction at negative electrode 13 by way of electrolyte 9. More specifically, electrochemical reactions such as the following are made to occur.

### <Reaction at Positive Electrode>

$${O}_{2} + 2 {H}_{2} O + 4 {e}^{-} →︀ 4 {OH}^{-}$$

### <Reaction at Negative Electrode>

$$Sn →︀ {Sn}^{2 +} + 2 {e}^{-}$$

To cause the foregoing electrochemical reaction(s) to occur efficiently, it is preferred that the area of the surface of negative electrode 13 be made to be not less than 20 times the area of the surface of positive electrode 12. In the context of the present specification, note that what is referred to as the “surfaces” of electrodes 12, 13 indicates those portions of electrodes 12, 13 that are in contact with electrolyte 9, this also being referred to as the “wetted surfaces” of electrodes 12, 13. It is therefore preferred, as is the case in the present embodiment, that the constitution of negative electrode 13 be such that it is formed so as to be cylindrical.

Because this will make it possible to cause the area of the surface of negative electrode 13 to be increased relative to the area of the surface of positive electrode 12, this will make it possible to cause the electrochemical reaction(s) to occur efficiently. Accordingly, it will, for example, be possible to improve the accuracy of measurement by oxygen sensor 5. What is more, because this will make it possible to increase the area of the surface of negative electrode 13 while at the same time suppressing increase in the volume of negative electrode 13, this will make it possible, for example, to suppress increase in the size of oxygen sensor 5.

Retainer 15 might, for example, be respectively secured to positive electrode 12 and negative electrode 13. A constitution may be adopted in which, e.g., as is the case in the present embodiment, retainer 15 is arranged at the interior of negative electrode 13, and an outside circumferential portion of retainer 15 is secured to an inside circumferential portion of negative electrode 13. Furthermore, a constitution may be adopted in which, e.g., as is the case in the present embodiment, retainer 15 is formed so as to be cylindrical, positive electrode 12 is formed so as to be columnar, positive electrode 12 is arranged at the interior of retainer 15, and an outside circumferential portion of positive electrode 12 is secured to an inside circumferential portion of retainer 15.

Electrode securing portion 16 might, for example, be respectively secured to negative electrode 13 and housing 14. A constitution may be adopted in which, e.g., as is the case in the present embodiment, electrode securing portion 16 is formed so as to be cylindrical, negative electrode 13 is arranged at the interior of electrode securing portion 16, and an outside circumferential portion of negative electrode 13 is secured to an inside circumferential portion of electrode securing portion 16. Furthermore, a constitution may be adopted in which, e.g., as is the case in the present embodiment, electrode securing portion 16 is arranged at the interior of housing 14, and an outside circumferential portion of electrode securing portion 16 is secured to an inside circumferential portion of housing 14.

Furthermore, to cause the foregoing electrochemical reaction(s) to occur efficiently, it is preferred that the surface of positive electrode 12 be arranged so as to be nearer than the surface of negative electrode 13 to permeable membrane 11. It is therefore preferred, as is the case in the present embodiment, that the constitution be such that the tip of retainer 15 protrudes beyond negative electrode 13, and such that the surface of positive electrode 12 is arranged at the tip of retainer 15.

Moreover, to cause the electrochemical reaction at positive electrode 12 to occur efficiently, it is preferred that the constitution be such that the surface of positive electrode 12 comes in contact with permeable membrane 11. Note, however, where it is said that “the surface of positive electrode 12 comes in contact with permeable membrane 11” that this should be understood to also include situations in which, e.g., due to capillary action, there is a small amount of electrolyte 9 present in a gap between permeable membrane 11 and the surface of positive electrode 12. In addition, it is even more preferred, as is the case in the present embodiment, that the constitution be such that the surface of positive electrode 12 comes in contact with and presses upon permeable membrane 11 so as to cause permeable membrane 11 which has elasticity to be made to undergo tension.

Note that the electrochemical reactions occurring at the respective electrodes 12, 13 are influenced by the temperatures at the respective electrodes 12, 13. Accordingly, where the difference between the temperature at positive electrode 12 and the temperature at negative electrode 13 is large, there is a possibility that there could be occurrence of a large error in the value of dissolved oxygen that is measured by oxygen sensor 5.

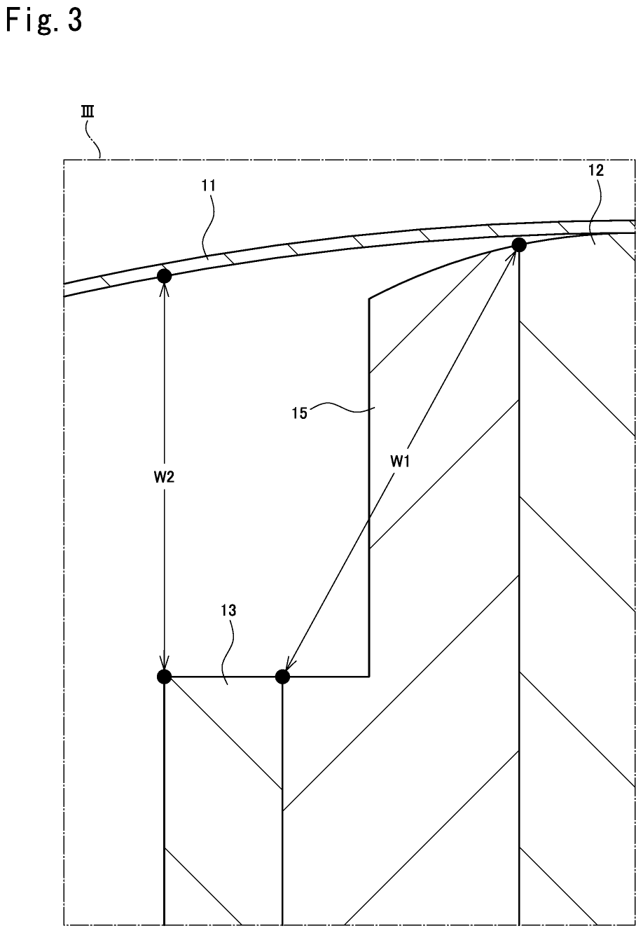

It is therefore preferred, as shown in FIG. 3, that the constitution be such that positive electrode 12 and negative electrode 13 are arranged in mutual proximity. It is therefore preferred, as is the case in the present embodiment, that the constitution be such that minimum distance W1 between the surface of positive electrode 12 and the surface of negative electrode 13 be not greater than 4.5 mm. This will make it possible to suppress occurrence of a difference between the temperature at positive electrode 12 and the temperature at negative electrode 13, intervening between which is electrolyte 9. At FIG. 3, note that electrolyte 9 is not shown.

Furthermore, because the temperature at positive electrode 12 and the temperature at negative electrode 13 will be affected by the temperature of the target liquid, it is preferred that a constitution be adopted in which negative electrode 13 is arranged so as to be near permeable membrane 11. It is therefore preferred, as is the case in the present embodiment, that the constitution be such that minimum distance W2 between permeable membrane 11 and the surface of negative electrode 13 be not greater than 4.0 mm. Because this will cause the temperature at positive electrode 12 and the temperature at negative electrode 13 to respectively approach the temperature of the target liquid, this will make it possible to suppress occurrence of a difference between the temperature at positive electrode 12 and the temperature at negative electrode 13.

It should be understood that there is no particular limitation with respect to the target liquid; for example, it may be tap water, drinking water, supply line water, sewer line water, water from rivers, lakes, marshes, or brackish estuaries, water containing industrial waste or from aquaculture tanks, manufacturing wastewater, wastewater from food factories or semiconductor manufacturing processes, human excreta, cooling water for air conditioning, and/or the like, and there are situations where it might be treated liquid (liquid in mid-treatment or liquid that has undergone treatment) from a treatment facility, or where it might be a test solution that has been placed within a container. In addition, there is no particular limitation with respect to the temperature of the target liquid, there being situations where this might, for example, be 0° C. or lower, or where this might be 40° C. or higher.

It is therefore preferred that the constitution be such that electrolyte 9 is water-soluble and contains inert polyol. More specifically, it is therefore preferred that the constitution be such that, among polyols, electrolyte 9 contain at least one of glycerol, erythritol, sorbitol, ethylene glycol, and/or propanediol.

Because this will cause the freezing point of electrolyte 9 to be made lower than 0° C., this will make it possible, even where the temperature of the target liquid is 0° C. or lower, to suppress freezing of electrolyte 9 when the temperature of the target liquid is a temperature that is greater than the freezing point of electrolyte 9. Accordingly, this will make it possible to properly measure the dissolved oxygen of a target liquid which is at low temperature. In addition, it is preferred, for example, that the freezing point of electrolyte 9 be −30° C. to −5° C.

Furthermore, where the concentration of salt contained within electrolyte 9 is adjusted, while it is possible that the freezing point effect might make it possible to lower the freezing point of electrolyte 9 to 0° C. or lower, because any solution will possess a certain solubility, it will only be possible for salt to be dissolved within electrolyte 9 to a concentration that is on the same order as or lower than the concentration of salt within the target liquid. That is, the freezing point of electrolyte 9 would be a temperature that is the same as or greater than the temperature of the freezing point of the target liquid. But where electrolyte 9 is made to contain polyol, even where the concentration of salt therein is on the same order as the concentration of salt within the target liquid, because, to the extent that it contains polyol, the freezing point of electrolyte 9 will be lower than the freezing point of the target liquid, it will be possible to properly measure the dissolved oxygen in the target liquid even at temperatures in the vicinity of the temperature at which the target liquid begins to freeze.

Note that it is preferred that the polarity (electrical bias present within the molecule) of polyol contained within electrolyte 9 be close to that of water. For example, polarity may be expressed by the solubility parameter (SP value). In addition, while there is no particular limitation with respect thereto, whereas the solubility parameter of water is 23.4 [(MPa)^{1/2}], it is preferred that the solubility parameter of polyol be, for example, not less than 11 [(MPa)^{1/2}]; further, it is even more preferred that this be, for example, not less than 14 [(MPa)^{1/2}]; and further, it is extremely preferred that this be, for example, not less than 16 [(MPa)^{1/2}]. This will make it possible to cause the foregoing electrochemical reaction(s) to occur efficiently.

For example, the solubility parameter of glycerol is 16.5 [(MPa)^{1/2}], the solubility parameter of ethylene glycol is 14.2 [(MPa)^{1/2}], and the solubility parameter of 1,3-propanediol is 11.5 [(MPa)^{1/2}]. Accordingly, among the various polyol substances, it is preferred that a constitution be adopted in which electrolyte 9 contains glycerol; and in particular, it is even more preferred that a constitution be adopted in which the polyol contained within electrolyte 9 is only glycerol. And it is also the case that while glycerol is water soluble, hygroscopic, and inert, it is nontoxic.

Furthermore, it is preferred that the specific volume of polyol in electrolyte 9 be chosen as appropriate. For example, where electrolyte 9 contains salt (e.g., NaOH, KOH, etc.), what is referred to as the specific volume of polyol in electrolyte 9 might be the “volume of polyol” expressed as a fraction of the “volume of electrolyte 9”.

For example, to suppress occurrence of a situation in which the specific volume of polyol within electrolyte 9 becomes too low, it is, for example, preferred that the specific volume of polyol within electrolyte 9 be not less than 10%, and it is, for example, even more preferred that this be not less than 20%. This will make it possible to suppress freezing of electrolyte 9 when measuring a target liquid that is at low temperature.

Furthermore, for example, to suppress occurrence of a situation in which the specific volume of polyol within electrolyte 9 becomes too high, it is, for example, preferred that the specific volume of polyol within electrolyte 9 be not greater than 70%, and it is, for example, even more preferred that this be not greater than 50%. Because this will make it possible to ensure that the specific volume of salt is adequate, this will make it possible to definitively cause occurrence of the foregoing electrochemical reaction(s) by way of electrolyte 9.

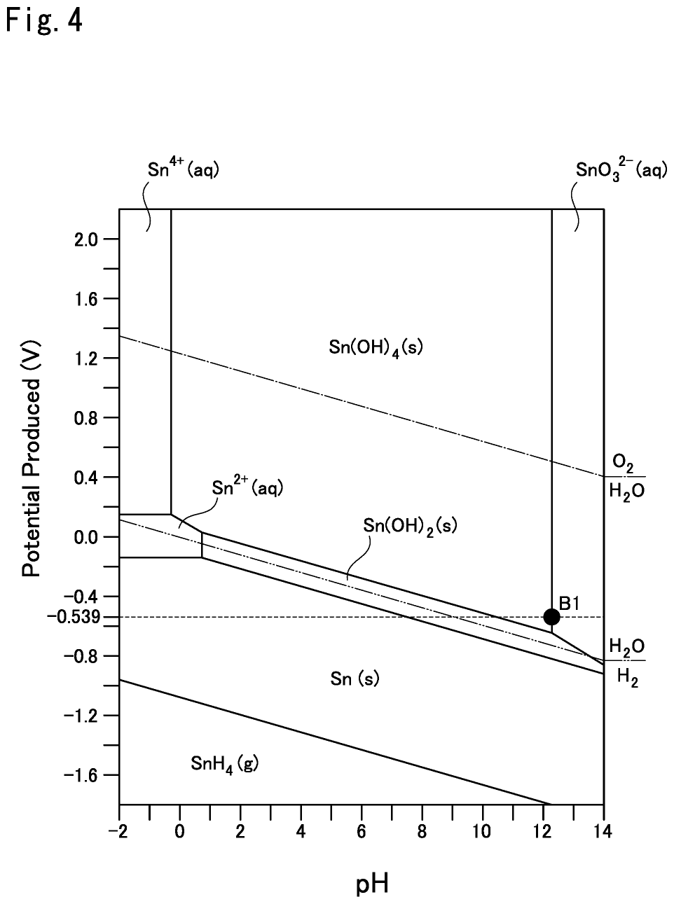

Here, a potential-pH diagram for an Sn—H₂O system (temperature=25° C.) in shown at FIG. 4. At FIG. 4, the horizontal axis is the pH of the aqueous solution, and the vertical axis is the potential relative to the NHE reference. In addition, at FIG. 4, the regions labeled as SnH₄ (g), Sn(s), Sn (OH)₂(s), Sn (OH)₄(s), Sn²⁺ (aq), Sn⁴⁺ (aq), and SnO₃²⁻ (aq) respectively show where these stably exist.

Thus, when negative electrode 13 is formed from tin, it can be understood from FIG. 4 which substance(s) stably exist, i.e., which substance(s) are generated in large quantities, based on the potential produced at negative electrode 13 (vertical axis) and the pH of electrolyte 9 (horizontal axis). Note that (g) indicates a gas, (s) indicates an insoluble solid, and (aq) indicates a water-soluble ion. Furthermore, at FIG. 4, the single-dot chain line is the boundary corresponding to redox equilibrium between water and oxygen, and the double-dot chain line is the boundary corresponding to redox equilibrium between water and hydrogen.

In addition, to cause the electrochemical reaction at positive electrode 12 to occur efficiently, it is preferred that the constitution be such that positive electrode 12 contain at least one of gold, silver, platinum, and/or carbon. In addition, when positive electrode 12 is formed from gold, silver, platinum, and/or carbon, and negative electrode 13 is formed from tin, electrochemical reactions such as the following are made to occur.

### <Reaction at Positive Electrode>

O₂+2H₂O+4e⁻=4OH⁻+0.401 V (standard redox potential at positive electrode)

### <Reaction at Negative Electrode>

Sn=Sn²⁺+2e⁻+0.138 V (standard redox potential at negative electrode)

Accordingly, the potential produced at negative electrode 13 will be −0.539 V, as indicated below.

$$Potential Produced at Negative Electrode 13 = - ( standard redox potential at positive electrode + standard redox potential at negative electrode ) = - ( 0.401 V + 0.138 V ) = - 0.539 V$$

At FIG. 4, the dashed line is a line indicating where the potential produced is −0.539 V. In addition, from FIG. 4, it can be estimated that the value of the pH at boundary B1 between the region in which stannic hydroxide [Sn(OH)₂] stably exists and the region in which stannate ion [SnO₃²⁻] stably exists is 12.2.

As a result, at oxygen sensor 5, stannic hydroxide [Sn (OH)₂], which is an insoluble solid, will stably exist when the pH of electrolyte 9 is less than 12.2. Accordingly, there is a possibility that stannic hydroxide will be produced, that stannic hydroxide will cover the surfaces of electrodes 12, 13 (particularly negative electrode 13), and that this will interfere with occurrence of the foregoing electrochemical reaction(s).

It is therefore preferred that the constitution be such that the pH of electrolyte 9 is not less than 12.2. Where this is the case, stannate ion [SnO₃²⁻], which is water soluble, will stably exist. Accordingly, because it will be possible to suppress production of stannic hydroxide, this will make it possible to suppress occurrence of situations in which stannic hydroxide might otherwise cover the surfaces of electrodes 12, 13.

As a result, because this will make it possible to suppress occurrence of situations which in occurrence of the electrochemical reaction(s) might otherwise be interfered with, this may make it possible, for example, to properly measure the amount of dissolved oxygen, and/or make it possible, for example, to increase the service life of oxygen sensor 5. Note that the value of the pH at boundary B1 between the region in which stannic hydroxide [Sn (OH)₂] stably exists and the region in which stannate ion [SnO₃²⁻] stably exists does not vary with the temperature of the aqueous solution.

A constitution in which the pH of electrolyte 9 is, for example, not less than 12.3 is more preferred; a constitution in which this is, for example, not less than 12.4 is even more preferred; and a constitution in which this is, for example, not less than 12.5 is extremely preferred. This will make it possible to suppress occurrence of situations in which stannic hydroxide [Sn (OH)₂], which is an insoluble solid, might otherwise exist. While there is no particular limitation with respect thereto, note, e.g., as is the case in the present embodiment, that the pH of electrolyte 9 may be not less than 12.8. Furthermore, while there is no particular limitation with respect thereto, the pH of electrolyte 9, e.g., as is the case in the present embodiment, may be not greater than 14.0.

Furthermore, in accordance with the present embodiment, the surface of positive electrode 12 comes in contact with permeable membrane 11. This will make it possible—even where stannic hydroxide [Sn (OH)₂], which is an insoluble solid, is produced—to suppress occurrence of a situation in which stannic hydroxide might otherwise enter the space between permeable membrane 11 and the surface of positive electrode 12. Accordingly, it will be possible to suppress occurrence of situations in which stannic hydroxide might otherwise cover the surface of positive electrode 12.

While there is no particular limitation with respect thereto, note that electrolyte 9 may contain buffering substance(s). This will make it possible to suppress fluctuation in the pH of electrolyte 9 as a result of the influence of acidic gases. While there is no particular limitation with respect thereto, note that phosphate buffer solutions, KCl—NaOH buffer solutions, and so forth may be cited as examples of buffer solutions that contain buffering substances.

It so happens, e.g., where negative electrode 13 contains zinc, that there are cases in which the products that are ultimately produced by the electrode reaction(s) will be zinc oxide (ZnO) (Zn→Zn²⁺→Zn (OH)₂ and ZnO). In such cases, if zinc oxide is arranged within polyol-containing electrolyte 9, there is a possibility that the zinc oxide will act as photocatalyst and will cause production of hydrogen.

In addition, if hydrogen is produced within electrolyte 9, because there will be swelling of permeable membrane 11, this will cause occurrence of gaps between permeable membrane 11 and positive electrode 12. This will make it impossible for dissolved oxygen in the target liquid to be properly measured by oxygen sensor 5. To address this, because an oxygen sensor 5 associated with the present embodiment is such that negative electrode 13 contains tin, the fact that it does not have catalytic function such as would cause production of hydrogen within electrolyte 9 means that it will be possible to suppress occurrence of gaps between permeable membrane 11 and positive electrode 12.

As described above, the water quality measuring device 1 according to this embodiment comprises the oxygen sensor 5.

And, as in the present embodiment, it is preferred that the oxygen sensor 5 comprises:
electrolyte 9;
a liquid-containing portion 10 that has an opening 10a and that contains the electrolyte 9 therewithin;
a permeable membrane 11 that has oxygen permeability and that covers the opening 10a; and
positive electrode 12 and negative electrode 13 that are arranged so as to come in contact with the electrolyte 9;
wherein the negative electrode 13 contains tin; and
wherein the electrolyte 9 contains polyol.

In accordance with such constitution, because electrolyte 9 contains polyol, the freezing point of electrolyte 9 will be lower than 0° C. This will make it possible to suppress freezing of electrolyte 9 when the temperature of the target liquid is a temperature that is greater than the freezing point of electrolyte 9. Accordingly, this will make it possible to properly measure the dissolved oxygen in a target liquid which is at low temperature.

Note, as in the present embodiment, it is preferred that the oxygen sensor 5 includes a configuration in which:
the polyol includes at least one of glycerol, erythritol, sorbitol, ethylene glycol, and propanediol.

Further, as in the present embodiment, it is preferred that the oxygen sensor 5 includes a configuration in which:
the positive electrode 12 contains at least one of gold, silver, platinum, and carbon; and
pH of the electrolyte 9 is not less than 12.2.

In accordance with such constitution, because negative electrode 13 contains tin, positive electrode 12 contains at least one of gold, silver, platinum, and carbon, and the pH of electrolyte 9 is not less than 12.2, the potential produced at negative electrode 13 will make it possible to suppress occurrence of a situation in which stannic hydroxide, which is an insoluble solid, might otherwise be produced. This will make it possible to suppress occurrence of situations in which stannic hydroxide might otherwise cover the surfaces of electrodes 12, 13.

Further, as in the present embodiment, it is preferred that the oxygen sensor 5 further comprises a retainer 15 that retains the positive electrode 12 and the negative electrode 13;
the negative electrode 13 is formed so as to be cylindrical;
the retainer 15 is arranged at an interior of the negative electrode 13 so as to cause a tip thereof to protrude beyond the negative electrode 13; and
a surface of the positive electrode 12 is arranged at the tip of the retainer 15.

In accordance with such constitution, because negative electrode 13 is formed so as to be cylindrical, it will be possible to cause the area of the surface of negative electrode 13 to be increased relative to the area of the surface of positive electrode 12, as a result of which it will be possible to cause the electrochemical reaction(s) to occur efficiently. And yet, because it will be possible to suppress increase in the volume of negative electrode 13, it will be possible to suppress increase in the size of oxygen sensor 5.

Further, as in the present embodiment, it is preferred that the oxygen sensor 5 includes a configuration in which:
a minimum distance W1 between the surface of the positive electrode 12 and a surface of the negative electrode 13 is not greater than 4.5 mm.

In accordance with such constitution, because minimum distance W1 between the surface of positive electrode 12 and the surface of negative electrode 13 is not greater than 4.5 mm, it will be possible to suppress occurrence of a difference between the temperature at positive electrode 12 and the temperature at negative electrode 13, intervening between which is electrolyte 9. This will make it possible to suppress occurrence of error when measuring the amount of dissolved oxygen.

Further, as in the present embodiment, it is preferred that the oxygen sensor 5 includes a configuration in which:
a minimum distance W2 between the permeable membrane 11 and the surface of the negative electrode 13 is not greater than 4.0 mm.

In accordance with such constitution, because minimum distance W2 between permeable membrane 11 and the surface of negative electrode 13 is not greater than 4.0 mm, this will cause the temperature at positive electrode 12 and the temperature at negative electrode 13 to respectively approach the temperature of the target liquid. Because this will make it possible to suppress occurrence of a difference between the temperature at positive electrode 12 and the temperature at negative electrode 13, this will make it possible to suppress occurrence of error when measuring the amount of dissolved oxygen.

The water quality measuring device 1, the oxygen sensor 5 and an oxygen measuring method are not limited to the configuration of the embodiment described above, and the effects are not limited to those described above. It goes without saying that the water quality measuring device 1, the oxygen sensor 5 and an oxygen measuring method can be variously modified without departing from the scope of the subject matter of the present invention. For example, the constituents, methods, and the like of various modified examples described below may be arbitrarily selected and employed as the constituents, methods, and the like of the embodiments described above, as a matter of course.

At oxygen sensor 5 associated with the foregoing embodiment, the constitution is such that negative electrode 13 is formed so as to be cylindrical. However, oxygen sensor 5 is not limited to such constitution. For example, it is also possible to adopt a constitution in which negative electrode 13 is formed so as to be planar.

### REFERENCE SIGNS LIST

1 water quality measuring device
2 detector
3 device main body
3a input unit
3b processor
3c output unit
4 communication mean
5 oxygen sensor
6 temperature sensor
7 detector main body
8 protective unit
8a opening
9 electrolyte
10 liquid-containing portion
10a opening
10b opening
11 permeable membrane
12 positive electrode
13 negative electrode
14 housing
15 retainer
16 electrode securing portion
17 membrane securing portion
18 seal

1. An oxygen sensor comprising:
electrolyte;
a liquid-containing portion that has an opening and that contains the electrolyte therewithin;
a permeable membrane that has oxygen permeability and that covers the opening; and
positive electrode and negative electrode that are arranged so as to come in contact with the electrolyte;
wherein the negative electrode contains tin; and
wherein the electrolyte contains polyol.

2. The oxygen sensor according to claim 1 wherein the polyol includes at least one of glycerol, erythritol, sorbitol, ethylene glycol, and propanediol.

3. The oxygen sensor according to claim 1 wherein
the positive electrode contains at least one of gold, silver, platinum, and carbon; and
pH of the electrolyte is not less than 12.2.

4. The oxygen sensor according to claim 1 wherein
the oxygen sensor further comprises a retainer that retains the positive electrode and the negative electrode;
the negative electrode is formed so as to be cylindrical;
the retainer is arranged at an interior of the negative electrode so as to cause a tip thereof to protrude beyond the negative electrode; and
a surface of the positive electrode is arranged at the tip of the retainer.

5. The oxygen sensor according to claim 4 wherein a minimum distance between the surface of the positive electrode and a surface of the negative electrode is not greater than 4.5 mm.

6. The oxygen sensor according to claim 4 wherein a minimum distance between the permeable membrane and the surface of the negative electrode is not greater than 4.0 mm.

7. The oxygen sensor according to claim 1 wherein the oxygen sensor is a galvanic cell-type sensor that causes an electric potential to be spontaneously produced between the positive electrode and the negative electrode.

8. A water quality measuring device comprising the oxygen sensor according to claim 1.

9. An oxygen measuring method wherein the oxygen sensor according to claim 1 is used to measure dissolved oxygen at a target liquid.

10. The oxygen sensor according to claim 4 wherein
the positive electrode is formed so as to be columnar;
the positive electrode is arranged at an interior of retainer; and
an outside circumferential portion of the positive electrode is secured to an inside circumferential portion of retainer.

11. The oxygen sensor according to claim 4 wherein a minimum distance between the permeable membrane and the surface of the negative electrode is less than minimum distance between the surface of the positive electrode and a surface of the negative electrode.

12. The oxygen sensor according to claim 4 wherein the surface of positive electrode comes in contact with the permeable membrane and presses upon the permeable membrane.

13. The oxygen sensor according to claim 4 wherein
the liquid-containing portion further comprises
a housing that is formed so as to be cylindrical; and
an electrode securing portion that causes the positive electrode and the negative electrode to be secured to the housing;
the electrode securing portion is formed so as to be cylindrical;
the negative electrode is arranged at an interior of electrode securing portion;
an outside circumferential portion of the negative electrode is secured to an inside circumferential portion of the electrode securing portion;
the electrode securing portion is arranged at an interior of the housing; and
an outside circumferential portion of electrode securing portion is secured to an inside circumferential portion of the housing.

14. The oxygen sensor according to claim 1 wherein a freezing point of the electrolyte is −30° C. to −5° C.

15. The oxygen sensor according to claim 1 wherein a specific volume of polyol within the electrolyte is not less than 10%.

16. An oxygen sensor comprising:
electrolyte;
a liquid-containing portion that has an opening and that contains the electrolyte therewithin;
a permeable membrane that has oxygen permeability and that covers the opening;
positive and negative electrodes that are arranged so as to come in contact with the electrolyte; and
a retainer that retains the positive electrode and the negative electrode;
the negative electrode is formed so as to be cylindrical;
the retainer is arranged at an interior of the negative electrode so as to cause a tip thereof to protrude beyond the negative electrode; and
a surface of the positive electrode is arranged at the tip of the retainer.

17. The oxygen sensor according to claim 16 wherein
the positive electrode is formed so as to be columnar;
the positive electrode is arranged at an interior of retainer; and
an outside circumferential portion of the positive electrode is secured to an inside circumferential portion of retainer.

18. The oxygen sensor according to claim 16 wherein a minimum distance between the permeable membrane and the surface of the negative electrode is less than minimum distance between the surface of the positive electrode and a surface of the negative electrode.

19. The oxygen sensor according to claim 16 wherein the surface of positive electrode comes in contact with the permeable membrane and presses upon the permeable membrane.

20. The oxygen sensor according to claim 16 wherein
the liquid-containing portion further comprises
a housing that is formed so as to be cylindrical; and
an electrode securing portion that causes the positive electrode and the negative electrode to be secured to the housing;
the electrode securing portion is formed so as to be cylindrical;
the negative electrode is arranged at an interior of electrode securing portion;
an outside circumferential portion of the negative electrode is secured to an inside circumferential portion of the electrode securing portion;
the electrode securing portion is arranged at an interior of the housing; and
an outside circumferential portion of electrode securing portion is secured to an inside circumferential portion of the housing.
